(19) United States
(12) Patent Application Publication
(10) Pub. No.: US 2023/0414667 A1
(43) Pub. Date: Dec. 28, 2023

(54) USE OF PROSTAGLANDIN E2 (PGE2)-PRIMED MESENCHYMAL STEM CELL (MSC) PRODUCT IN PREPARATION OF DRUG FOR TREATING LUNG INJURY (LI)

(71) Applicant: NANKAI UNIVERSITY, Tianjin (CN)

(72) Inventors: Zongjin LI, Tianjin (CN); Zihan CHAI, Tianjin (CN); Chen WANG, Tianjin (CN); Kamal HEZAM, Tianjin (CN)

(73) Assignee: NANKAI UNIVERSITY, Tianjin (CN)

(21) Appl. No.: 18/212,404

(22) Filed: Jun. 21, 2023

(30) Foreign Application Priority Data
Jun. 24, 2022 (CN) 202210721593.5

(51) Int. Cl. A61K 35/28; C12N 5/0775

(52) U.S. Cl. CPC A61K 35/28; C12N 5/0663; C12N 2501/02

(57) ABSTRACT

The present disclosure relates to the technical field of medicine, in particular to use of a prostaglandin E2 (PGE2)-primed mesenchymal stem cell (MSC) product in preparation of a drug for treating lung injury (LI). A preparation method of the PGE2-primed MSC product includes conducting mixed culture on PGE2 and MSCs to obtain the PGE2-primed MSC product. The PGE2 and the MSCs are subjected to the mixed culture to enhance functions of the MSCs, promote migration of the MSCs to injury sites, strengthen anti-inflammatory effects of the MSCs, facilitate MSC proliferation, effectively relieve various lung injuries, and accelerate regeneration of injured alveolar epithelial cells. The drug prepared from the PGE2-primed MSC product provided by the present disclosure can effectively relieve LI and accelerate the regeneration of injured alveolar epithelial cells, and a remarkable treatment effect is obtained.

### CROSS REFERENCE TO RELATED APPLICATION

This patent application claims the benefit and priority of Chinese Patent Application No. 202210721593.5, filed with the China National Intellectual Property Administration on Jun. 24, 2022, the disclosure of which is incorporated by reference herein in its entirety as part of the present application.

### TECHNICAL FIELD

The present disclosure relates to the technical field of medicine, in particular to use of a prostaglandin E2 (PGE2)-primed mesenchymal stem cell (MSC) product in preparation of a drug for treating lung injury (LI).

### BACKGROUND

Lung injury includes acute lung injury (ALI), acute respiratory distress syndrome (ARDS), pulmonary fibrosis, silicosis, and radiation-induced LI. ALI refers to the damage of alveolar epithelial cells and pulmonary capillary endothelial cells caused by non-cardiogenic causes, which can lead to pulmonary inflammatory cell infiltration and pulmonary interstitial edema. ARDS is an acute respiratory failure caused by the enhanced permeability of alveolar capillaries caused by ALI. Patients have high mortality, and more effective drugs to control symptoms are urgently needed in clinical treatment. Pulmonary fibrosis is an irreversible diffuse interstitial lung disease that reduces alveolar ventilation and eventually leads to organ failure. Silicosis is the most common pneumoconiosis in the world, which is a disease characterized by extensive nodular fibrosis in the lung tissue due to the long-term inhalation of substantial free silica dust that accumulates in the lungs. Silicosis is a serious and disabling occupational disease with high morbidity in China at present. Radiation-induced LI is LI caused after thoracic radiation therapy. Radiation-induced LI is the most serious complication of radiotherapy, which manifests as pulmonary hemorrhage or increased alveolar fibrin exudation and tends to finally form pulmonary interstitial fibrosis. At present, there is no good clinical treatment to deal with various acute and chronic lung injuries. Although lung transplantation is an alternative treatment strategy, it is limited by the source of donors and high costs. Therefore, there is an urgent need to develop innovative and effective therapies to alleviate LI while promoting regeneration of injured alveolar epithelial cells.

### SUMMARY

To solve the above problems, the present disclosure provides use of a PGE2-primed MSC product in preparation of a drug for treating LI. The drug prepared from the PGE2-primed MSC product provided by the present disclosure can effectively relieve LI and accelerate the regeneration of injured alveolar epithelial cells, and a remarkable treatment effect is obtained.

To achieve the above objective, the present disclosure provides the following technical solutions:

The present disclosure provides use of a PGE2-primed MSC product in preparation of a drug for treating LI. A preparation method of the PGE2-primed MSC product includes conducting mixed culture on PGE2 and MSCs to obtain the PGE2-primed MSC product.

Preferably, the mixed culture is conducted for 12-24 h.

Preferably, the PGE2-primed MSC product includes PGE2-primed MSCs and/or exosomes secreted by the PGE2-primed MSCs.

Preferably, a culture medium for the mixed culture is based on Dulbecco's Modified Eagle Medium/Nutrient Mixture F-12 (DMEM/F12) and further supplemented with components of the following concentrations: 100 mL/L fetal bovine serum (FBS), 10 g/L L-glutamine, 10 mL/L non-essential amino acid solution, and 10 mL/L penicillin-streptomycin mixture.

Preferably, the MSC is one or more selected from the group consisting of placental-derived mesenchymal stem cell (PMSC), limbal stem cell (LSC), bone marrow mesenchymal stem cell (BMMSC), adipose mesenchymal stem cell (AMSC), umbilical cord mesenchymal stem cell (UCMSC), urine-derived stem cell (USC), endothelial progenitor cell (EPC), and cardiac stem cell (CSC).

Preferably, the LI is one or more selected from the group consisting of ALI, ARDS, pulmonary fibrosis, silicosis, and radiation-induced LI.

The present disclosure further provides a drug for treating LI. The drug includes a PGE2-primed MSC product. A preparation method of the PGE2-primed MSC product includes conducting mixed culture on PGE2 and MSCs to obtain the PGE2-primed MSC product.

Preferably, the mixed culture is conducted for 12-24 h.

The present disclosure has the following beneficial effects:

The present disclosure provides use of a PGE2-primed MSC product in preparation of a drug for treating LI. A preparation method of the PGE2-primed MSC product includes conducting mixed culture on PGE2 and MSCs to obtain the PGE2-primed MSC product. The PGE2 and the MSCs are subjected to the mixed culture. The MSCs are primed by the PGE2, thereby enhancing anti-inflammatory and cell regeneration-promoting functions of the MSCs, promoting migration of the MSCs to injury sites, strengthening anti-inflammatory effects of the MSCs, facilitating MSC proliferation, effectively relieving various lung injuries, and accelerating regeneration of injured alveolar epithelial cells. The drug prepared from the PGE2-primed MSC product provided by the present disclosure can effectively relieve LI and accelerate the regeneration of injured alveolar epithelial cells, and a remarkable treatment effect is obtained.

### BRIEF DESCRIPTION OF THE DRAWINGS

To illustrate the examples of the present disclosure or the technical solutions in the prior art more clearly, the accompanying drawings required in the examples will be briefly introduced below.

FIGS. 1A-B illustrate the morphology of MSCs before and after PGE2 priming;

FIGS. 2A-B illustrate gene expression levels of inflammatory factors and anti-fibrotic factors related to MSCs before and after PGE2 priming; * indicates a comparison with an MSCs-treated group;

FIG. 3 illustrates HE staining results of lung pathological sections, in which the right image of each group is an enlarged view of a box area in the left image; the scale on the left image is 200 μm, and that on the right image is 100 μm;

FIG. 4 illustrates Giemsa staining results of bronchoalveolar lavage fluid (BALF); the scale on the left image is 200 μm, and that on the right image is 100 μm; and

FIGS. 5A-D illustrate the gene expression of inflammatory factors after PGE2-primed MSCs treat LI;

MSCs represent MSCs before priming, and PEG2-MSCs represent MSCs after priming; and

Sham represents a normal group, PBS represents a PBS group, ALI represents acute LI; #indicates a comparison with the PBS group; and & indicates a comparison with the MSCs-treated group.

### DETAILED DESCRIPTION OF THE EMBODIMENTS

The present disclosure provides use of a PGE2-primed MSC product in preparation of a drug for treating LI.

In the present disclosure, the PGE2-primed MSC product preferably includes PGE2-primed MSCs and/or exosomes secreted by the PGE2-primed MSCs.

In the present disclosure, a preparation method of the PGE2-primed MSC product includes conducting mixed culture on PGE2 and MSCs to obtain the PGE2-primed MSC product.

In the present disclosure, the mixed culture is preferably conducted for 12-24 h, further preferably 12-18 h, and more preferably 12 h. In the present disclosure, when the PGE2 and the MSCs are subjected to the mixed culture, a concentration of the PGE2 in a culture medium is preferably 2-10 μmol/L, further preferably 2-5 μmol/L, and more preferably 2 μmol/L.

In the present disclosure, a culture medium for the mixed culture is based on DMEM/F12 and further preferably supplemented with components of the following concentrations: 100 mL/L FBS, 10 g/L L-glutamine, 10 mL/L non-essential amino acid solution, and 10 mL/L penicillin-streptomycin mixture.

The present disclosure can improve the anti-inflammatory ability of the MSCs by conducting the mixed culture on the PGE2 and the MSCs and priming the MSCs with the PGE2, and can promote LI repair by reducing inflammatory responses in various LI models after injury.

In the present disclosure, a preparation method of the exosomes secreted by the PGE2-primed MSCs preferably includes the following steps: culturing to obtain a supernatant according to the above method; subjecting the supernatant to a first centrifugation at 500 g for 10 min at 4° C., followed by a second centrifugation at 2000 g for 10 min at 4° C.; filtering the supernatant with a 0.22 μm filter; placing a filtered supernatant in an ultracentrifuge tube and conducting a third centrifugation at 120,000 g for 2 h at 4° C. to obtain exosome pellets, and resuspending the exosome pellets with phosphate buffered saline (PBS) to obtain primed exosomes.

In the present disclosure, the first centrifugation may remove cell debris; the second centrifugation may remove apoptotic bodies and other substances; and the third centrifugation may remove microvesicles with a diameter greater than 200 nm.

In the present disclosure, the MSC is preferably one or more selected from the group consisting of PMSC, LSC, BMMSC, AMSC, UCMSC, USC, EPC, and CSC.

In the present disclosure, the LI is preferably one or more selected from the group consisting of ALI, ARDS, pulmonary fibrosis, silicosis, and radiation-induced LI.

The present disclosure further preferably provides use of a PGE2-primed MSC product in preparation of a drug for inhibiting the expression of a pro-inflammatory factor and/or promoting the expression of an anti-inflammatory factor.

In the present disclosure, the preparation method of the PGE2-primed MSC product is the same as the above solution, and will not be repeated herein.

In the present disclosure, the pro-inflammatory factor is preferably one or more selected from the group consisting of tumor necrosis factor alpha (TNF-α), interleukin-6 (IL-6), and interleukin-1 beta (IL-1β); and the anti-inflammatory factor includes interleukin-10 (IL-10).

The present disclosure further provides a drug for treating LI, and the drug includes a PGE2-primed MSC product.

In the present disclosure, the preparation method of the PGE2-primed MSC product is the same as the above solution, and will not be repeated herein.

In order to further illustrate the present disclosure, the use of a PGE2-primed MSC product in preparation of a drug for treating LI provided by the present disclosure will be described in detail below with reference to the examples, but they should not be construed as limiting the protection scope of the present disclosure.

Sera used for cell culture in the following examples are purchased from Hyclone Laboratories Inc.; cell culture medium, double antibody, trypsin, glutamine are purchased from Gibco Inc.; and consumables required for cell culture are purchased from Corning Inc.

The experimental procedure of cell biology experiments including cell subculture, cryopreservation, and thawing is carried out with reference to the Culture of Animal Cells (6th Edition).

### Example 1

PGE2-Primed MSCs and Collection:

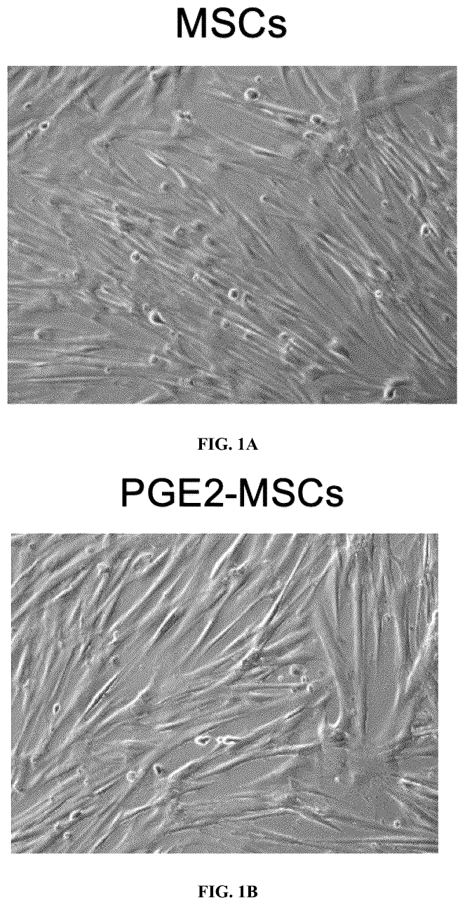

PMSCs at P3 to P5 were seeded in 6-well plates at a density of 5×10⁴ cells per well. After 24 h, when the cells grew in a good state, the culture medium was replaced with complete medium supplemented with PGE2 (the concentration of PGE2 in complete medium was 2 μmol/L). The cells were treated with PGE2 for 12 h and digested for collection. The complete medium was based on DMEM/F12 and only further supplemented with components of the following concentrations: 100 mL/L FBS, 10 g/L L-glutamine, 10 mL/L non-essential amino acid solution, and 10 mL/L penicillin-streptomycin mixture. The morphology of PGE2-primed MSCs was observed under an optical microscope. The results are shown in FIGS. 1A-B.

It can be seen from FIGS. 1A-B that there is no significant difference in the morphology of MSCs before and after priming, indicating that PGE2 priming has no significant effect on the phenotype of PMSCs.

### Example 2

Method for Extracting Exosomes (EVs) from PGE2-Primed MSCs:
1) Preparation of exosome-depleted serum: FBS was placed in an ultracentrifugation tube and centrifuged at 120,000 g for 2 h at 4° C.; the supernatant (exosomes were pellets) was extracted in a clean bench, filtered with a 0.22 μm filter, and stored in a −80° C. freezer for use.
2) PMSCs at P3 to P5 were seeded in a T75 flask at a density of 3×10⁶ cells per flask. After 24 h, when the cells grew in a good state, the culture medium was replaced with the complete medium supplemented with PGE2 described in Example 1. After PGE2 treatment for 12 h, when the cells were in the logarithmic growth phase and the cells reached 80% confluence, the complete medium was removed and the cells were washed twice with PBS. After washing, the conditioned medium was added to the flask for culture. After culture for 24 h, the conditioned medium was collected into a 50 mL centrifuge tube. The conditioned medium was based on DMEM/F12 and only further supplemented with components of the following concentrations: 100 mL/L exosome-depleted serum prepared in step 1), 10 g/L L-glutamine, 10 mL/L non-essential amino acid solution, and 10 mL/L penicillin-streptomycin mixture.
3) Exosome extraction (gradient centrifugation method): The supernatant of the conditioned medium collected in step 2) was centrifuged at 500 g for 10 min at 4° C. to remove cell debris and at 2,000 g for 10 min at 4° C. to remove apoptotic bodies and the like; the supernatant was filtered with a 0.22 μm filter to remove microvesicles with a diameter larger than 200 nm; the filtered supernatant was placed in an ultracentrifuge tube and centrifuged at 120,000 g for 2 h at 4° C. to obtain exosome pellets, and the exosome pellets were resuspended with PBS to obtain primed exosomes.

### Example 3

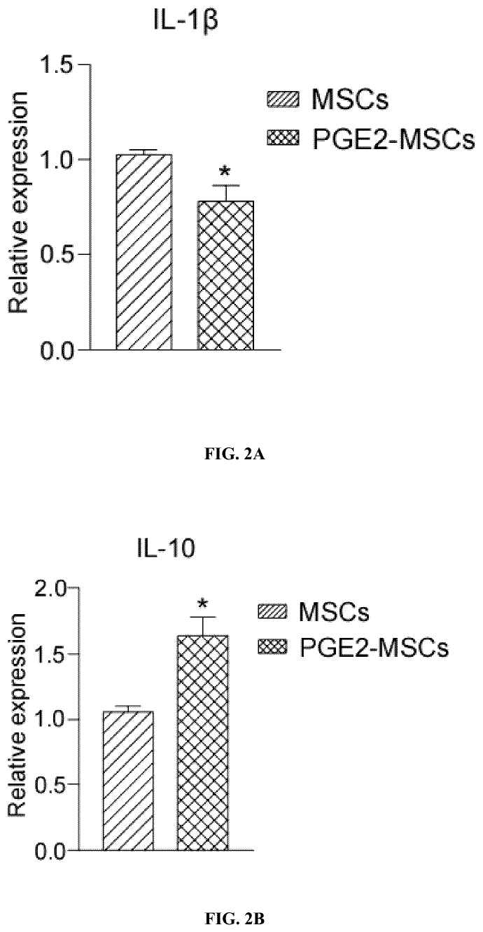

The changes in gene expression of related inflammatory factor IL-1β and anti-fibrotic factor IL-10 in unprimed MSCs and PGE2-primed MSCs prepared in Example 1 were detected by real-time fluorescence quantitative PCR. The experimental results are shown in FIGS. 2A-B, where the relative expression level of IL-1β in ordinary MSCs was 1, and that of IL-1β in PGE2-primed MSCs was 0.78; the relative expression level of IL-10 in ordinary MSCs was 1, and that of IL-10 in PGE2-primed MSCs was 1.64.

As can be seen from FIGS. 2A-B, after PGE2 treatment for 12 h, the expression level of the inflammatory factor IL-1β gene in the PMSCs is significantly downregulated, and that of the anti-fibrotic factor IL-10 gene is significantly upregulated. Visibly, PGE2-primed MSCs can improve the anti-inflammatory function of MSCs (downregulate the expression of the inflammatory factor and upregulate the expression of the anti-inflammatory factor).

### Example 4

Construction methods of mouse models of ALI/ARDS, pulmonary fibrosis and silicosis, and PGE2-primed MSC-based treatment method.

Construction Method of Mouse Models of ALI:

Forty 8-week-old C57/B6 male mice were weighed and anesthetized by intraperitoneal injection of 2.5% Avertin (0.2 mL/10 g body weight). Each mouse was immobilized on a small animal operating table in a supine position. The hair on the neck was depilated, the remaining hair was removed again with a depilatory cream, and the skin was disinfected with iodophor. The skin (about 0.5 cm long) was cut off along the midline of the neck, the submandibular glands were separated from the muscles surrounding the trachea, and the trachea was exposed. Subsequently, tracheal intubation was performed, and ALI was induced in each mouse by instillation of 5 mg/kg lipopolysaccharide (LPS, dissolved in PBS), by mouse weight. After successful modeling, they were randomly divided into four groups: a sham group, a PBS group, an MSCs-treated group, and a PGE2-primed MSCs-treated group. Herein, the sham group did not receive treatment, each mouse in the PBS group was injected with 250 μL of PBS, each mouse in the MSCs-treated group was treated with 1×10⁶ ordinary MSCs, and each mouse in the PGE2-primed MSCs-treated group was treated with 1×10⁶ PGE2-primed MSCs.

Construction Method of Mouse Models of Pulmonary Fibrosis:

Forty 8-week-old C57/B6 male mice were weighed and anesthetized by intraperitoneal injection of 2.5% Avertin (0.2 mL/10 g body weight). Each mouse was immobilized on a small animal operating table in a supine position. The hair on the neck was depilated, the remaining hair was removed again with a depilatory cream, and the skin was disinfected with iodophor. The skin (about 0.5 cm long) was cut off along the midline of the neck, the submandibular glands were separated from the muscles surrounding the trachea, and the trachea was exposed. Subsequently, tracheal intubation was performed, and pulmonary fibrosis was induced in each mouse by injection of 5 mg/kg bleomycin (dissolved in 100 μL of normal saline), by mouse weight. After successful modeling, they were randomly divided into four groups: a sham group, a PBS group, an MSCs-treated group, and a PGE2-primed MSCs-treated group. Herein, the sham group did not receive treatment, each mouse in the PBS group was injected with 250 μL of PBS, each mouse in the MSCs-treated group was treated with 1×10⁶ ordinary MSCs, and each mouse in the PGE2-primed MSCs-treated group was treated with 1×10⁶ PGE2-primed MSCs.

Construction Method of Mouse Models of Silicosis:

Forty 8-week-old C57/B6 male mice were weighed and anesthetized by intraperitoneal injection of 2.5% Avertin (0.2 mL/10 g body weight). Each mouse was immobilized on a small animal operating table in a supine position. The hair on the neck was depilated, the remaining hair was removed again with a depilatory cream, and the skin was disinfected with iodophor. The skin (about 0.5 cm long) was cut off along the midline of the neck, the submandibular glands were separated from the muscles surrounding the trachea, and the trachea was exposed. Tracheal intubation was performed, and silicosis was induced in each mouse by intratracheal injection of 50 μL of SiO₂ suspension (10⁵ mg/L). After successful modeling, they were randomly divided into four groups: a sham group, a PBS group, an MSCs-treated group, and a PGE2-primed MSCs-treated group. Herein, the sham group did not receive treatment, each mouse in the PBS group was injected with 250 μL of PBS, each mouse in the MSCs-treated group was treated with 1×10⁶ ordinary MSCs, and each mouse in the PGE2-primed MSCs-treated group was treated with 1×10⁶ PGE2-primed MSCs.

Construction Method of Mouse Models of Radiation-Induced LI:

Forty 8-week-old C57/B6 male mice were weighed and anesthetized by intraperitoneal injection of 2.5% Avertin (0.2 mL/10 g body weight). The anesthetized mice were exposed to thoracic irradiation. The irradiation source was GC40E W/2 C-440 γ-ray, and the irradiation dose was 20 Gy. After successful modeling, they were randomly divided into four groups: a sham group, a PBS group, an MSCs-treated group, and a PGE2-primed MSCs-treated group. Herein, the sham group did not receive treatment, each mouse in the PBS group was injected with 250 μL of PBS, each mouse in the MSCs-treated group was treated with 1×10⁶ ordinary MSCs, and each mouse in the PGE2-primed MSCs-treated group was treated with 1×10⁶ PGE2-primed MSCs.

In the construction methods of the above four models, the method for injecting different reagents into the mouse-tail vein is as follows:
step 1, a mouse was immobilized on a mouse immobilizer, and the tail was held naturally outwards to facilitate operations;
step 2, warm water or alcohol cotton balls were prepared in advance to wipe the tail of the mouse to slightly dilate the blood vessels in the tail, in order to facilitate the injection;
step 3, the skin with clear and thin blood vessels in the lower ⅓ to ½ of the tail was selected, and the angle of the syringe was adjusted to inject reagents, during which the group treated with ordinary MSCs or PGE2-primed MSCs was required to resuspend the cells with PBS in advance; and
step 4, successful injection into the tail vein was obtained once the injection was unobstructed or blood returning was visible; after the injection, hemostasis was obtained by gently pressing the injected site with a cotton swab, which was released until there was no bleeding; and the mouse was removed from the immobilizer and returned to its cage.

### Example 5

Pathological Analysis Methods for the Evaluation of LI and Repair Function.

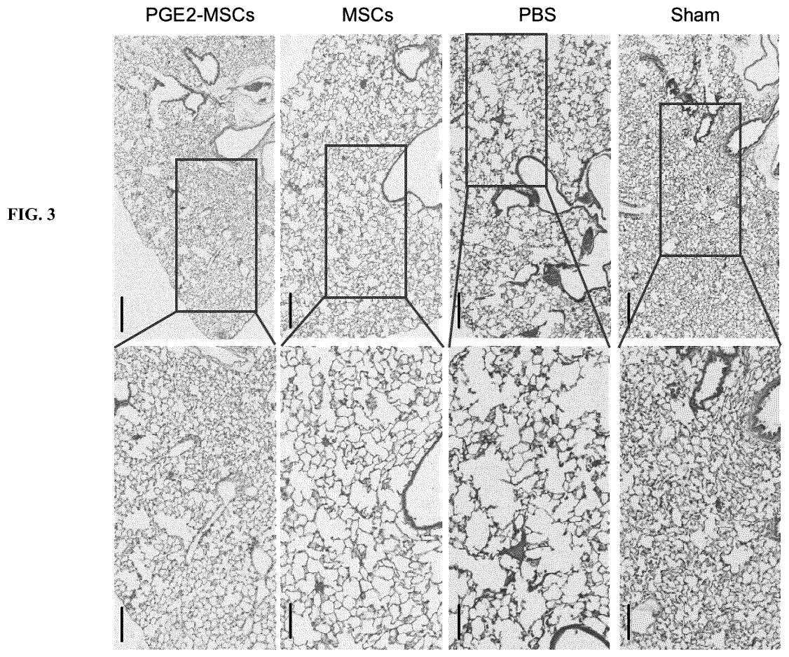

The structure-function analysis of tissue damage and repair was evaluated by hematoxylin-eosin (H&E) staining. In the model of ALI constructed in Example 2, after the mice were modeled, they were treated with PGE2-primed MSCs, sampled 24 h after the treatment. The tissue was fixed, dehydrated, paraffin-embedded, and paraffin-sectioned. The well-sectioned lung tissue sections were subjected to H&E staining to evaluate the degree of tissue damage and inflammatory infiltration. The results are shown in FIG. 3.

As can be seen from FIG. 3, PGE2-primed MSCs can effectively treat ALI, and the primed MSCs have better therapeutic potential than ordinary MSCs.

### Example 6

Detection Method of Pulmonary Inflammatory Response after MSCs-Based Treatment.

BALFs were collected from four groups of mouse model of ALI in Example 4 for Giemsa staining, so as to detect the pulmonary inflammatory response after treatment with PGE2-primed MSCs or exosomes thereof.

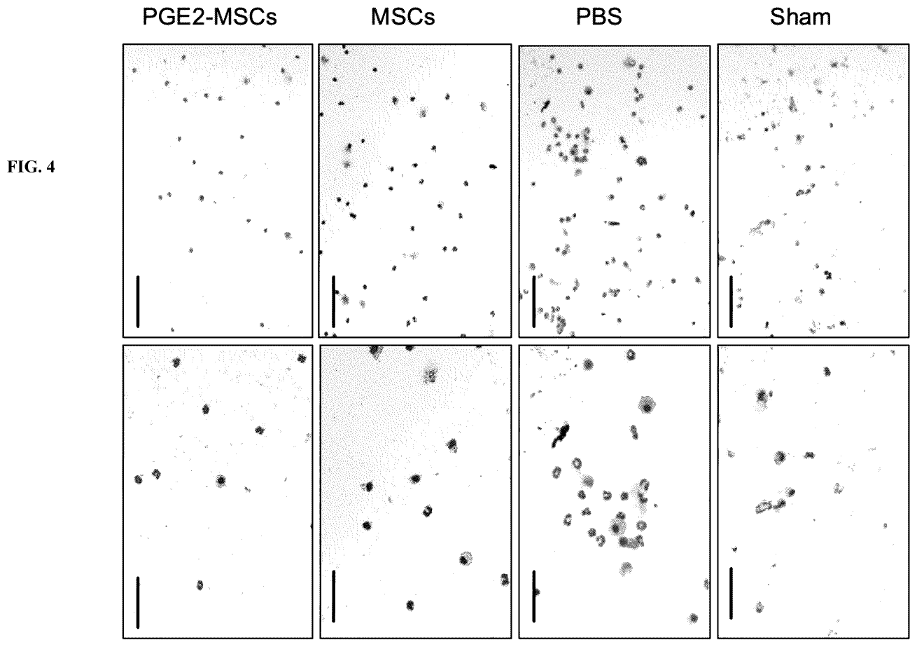

Collection method of BALFs: A mouse was anesthetized and immobilized on a small animal operating table in a supine position. The skin of the neck of the mouse was cut, and the peritracheal tissues were separated to expose the trachea of the mouse. After aspirating 0.3 mL of PBS, tracheal intubation was done at the upper end of the trachea, and 0.3 mL of PBS was pushed into the lung for lavage. The lavage was repeated three times and the BALF was collected into a 1.5 mL EP tube. The recovered BALF was centrifuged at 1,500 rpm for 10 min at 4° C., and the supernatant was collected and stored at −20° C. After preparation, the pulmonary inflammatory response was detected by Giemsa staining. The staining results are shown in FIG. 4. The results show that PGE2-primed MSCs can reduce the inflammatory response after LI.

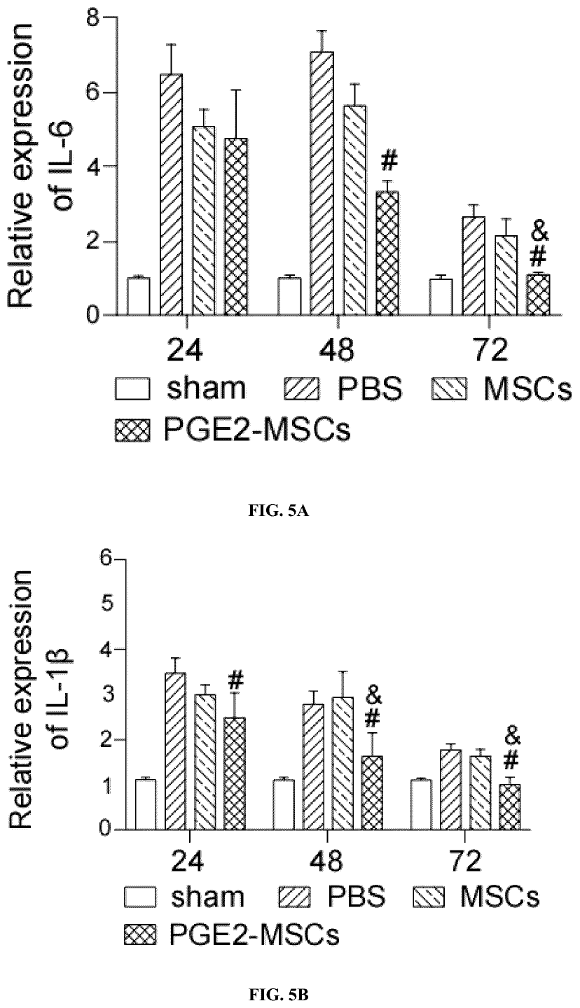

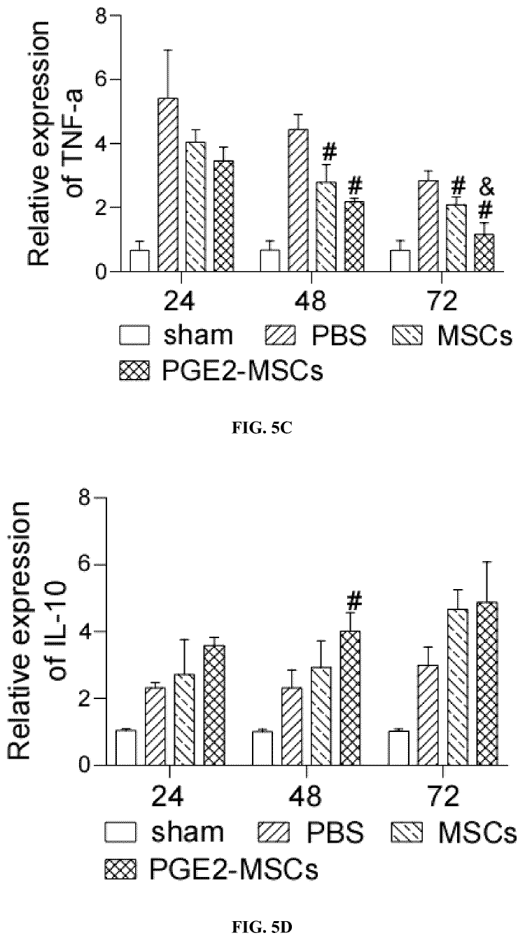

The changes in gene expression of inflammatory factors in the lungs after treatment with PGE2-primed MSCs were detected by real-time fluorescence quantitative PCR. The lung tissue of the mouse was sampled, and the RNA was extracted from the tissue by TRIzol method and reverse transcribed to obtain cDNA. The expression levels of inflammation-related genes were detected by real-time PCR. Real-time PCR results are shown in FIGS. 5A-D.

It can be seen from FIGS. 5A-D that treatment with PGE2-primed MSCs can better inhibit the expression of inflammatory factors in the lungs and promote the expression of the anti-fibrotic factor IL-10 gene.

In conclusion, the drug prepared from the PGE2-primed MSC product provided by the present disclosure can not only have excellent anti-inflammatory function and effectively relieve LI, but also can accelerate the regeneration of injured alveolar epithelial cells, and a remarkable treatment effect is obtained.

Although the present disclosure has been set forth as above in preferred examples, they are not intended to limit the present disclosure. Those skilled in the art can make various alterations and modifications without departing from the spirit and scope of the present disclosure. Therefore, the protection scope of the present disclosure should be subject to the protection scope defined by the claims.

What is claimed is:

1. A method for treating lung injury (LI), wherein a prostaglandin E2 (PGE2)-primed mesenchymal stem cell (MSC) drug is administered to a subject with LI; and a preparation method of the PGE2-primed MSC product comprises conducting mixed culture on PGE2 and MSCs to obtain the PGE2-primed MSC product.

2. The method according to claim 1, wherein the mixed culture is conducted for 12-24 h.

3. The method according to claim 1, wherein the PGE2-primed MSC product comprises PGE2-primed MSCs and/or exosomes secreted by the PGE2-primed MSCs.

4. The method according to claim 2, wherein the PGE2-primed MSC product comprises PGE2-primed MSCs and/or exosomes secreted by the PGE2-primed MSCs.

5. The method according to claim 1, wherein a culture medium for the mixed culture is based on Dulbecco's Modified Eagle Medium/Nutrient Mixture F-12 (DMEM/F12) and further supplemented with components of the following concentrations: 100 mL/L fetal bovine serum (FBS), 10 g/L L-glutamine, 10 mL/L non-essential amino acid solution, and 10 mL/L penicillin-streptomycin mixture.

6. The method according to claim 2, wherein a culture medium for the mixed culture is based on Dulbecco's Modified Eagle Medium/Nutrient Mixture F-12 (DMEM/F12) and further supplemented with components of the following concentrations: 100 mL/L fetal bovine serum (FBS), 10 g/L L-glutamine, 10 mL/L non-essential amino acid solution, and 10 mL/L penicillin-streptomycin mixture.

7. The method according to claim 1, wherein the MSC is one or more selected from the group consisting of placental-derived mesenchymal stem cell (PMSC), limbal stem cell (LSC), bone marrow mesenchymal stem cell (BMMSC), adipose mesenchymal stem cell (AMSC), umbilical cord mesenchymal stem cell (UCMSC), urine-derived stem cell (USC), endothelial progenitor cell (EPC), and cardiac stem cell (CSC).

8. The method according to claim 2, wherein the MSC is one or more selected from the group consisting of placental-derived mesenchymal stem cell (PMSC), limbal stem cell (LSC), bone marrow mesenchymal stem cell (BMMSC), adipose mesenchymal stem cell (AMSC), umbilical cord mesenchymal stem cell (UCMSC), urine-derived stem cell (USC), endothelial progenitor cell (EPC), and cardiac stem cell (CSC).

9. The method according to claim 1, wherein the LI is one or more selected from the group consisting of acute lung injury (ALI), acute respiratory distress syndrome (ARDS), pulmonary fibrosis, silicosis, and radiation-induced LI.

10. The method according to claim 2, wherein the LI is one or more selected from the group consisting of acute lung injury (ALI), acute respiratory distress syndrome (ARDS), pulmonary fibrosis, silicosis, and radiation-induced LI.

11. A drug for treating LI, wherein the drug comprises a PGE2-primed MSC product, and a preparation method of the PGE2-primed MSC product comprises conducting mixed culture on PGE2 and MSCs to obtain the PGE2-primed MSC product.

12. The drug according to claim 11, wherein the mixed culture is conducted for 12-24 h.
